(19) United States
(12) Patent Application Publication
(10) Pub. No.: US 2008/0177316 A1
(43) Pub. Date: Jul. 24, 2008

(54) APPARATUS AND METHODS FOR SPINAL IMPLANT

(76) Inventors: Brian J. Bergeron, Austin, TX (US); Charles R. Forton, Leander, TX (US); Abhijeet B. Joshi, Austin, TX (US)

Correspondence Address: PAUL D. YASGER;ABBOTT LABORATORIES, 100 ABBOTT PARK ROAD, DEPT. 377/AP6A, ABBOTT PARK, IL, 60064-6008, US

(21) Appl. No.: 11/564,930

(22) Filed: Nov. 30, 2006

(51) Int. Cl. A61B 17/70

(52) U.S. Cl. 606/254; 606/257

(57) ABSTRACT

A fixation or implant system (10) is provided for supporting a spinal column (12) and includes a pair of dynamic spinal rods (14, 16) that are fixed on laterally opposite sides of the spine (12). The rods (14,16) are configured to allow an initial range of spinal motion and to resist spinal motion beyond the initial range.

### CROSS-REFERENCE TO RELATED APPLICATIONS

Not Applicable.

### FEDERALLY SPONSORED RESEARCH OR DEVELOPMENT

Not Applicable.

### MICROFICHE/COPYRIGHT REFERENCE

Not Applicable.

### TECHNICAL FIELD

This invention relates generally to spinal implants, and more particularly to spinal implants or rods that allow extension and flexion of the spine.

### BACKGROUND OF THE INVENTION

Modern spine surgery often involves spinal fixation through the use of spinal implants or fixation systems to correct or treat various spine disorders or to support the spine. Spinal implants may help, for example, to stabilize the spine, correct deformities of the spine, facilitate fusion, or treat spinal fractures. A spinal fixation system typically includes corrective spinal instrumentation that is attached to selected vertebra of the spine by screws, hooks, and clamps. The corrective spinal instrumentation includes spinal rods or plates that are generally parallel to the patient's back. The corrective spinal instrumentation may also include transverse connecting rods that extend between neighboring spinal rods. Spinal fixation systems are used to correct problems in the cervical, thoracic, and lumbar portions of the spine, and are often installed posterior to the spine on opposite sides of the spinous process and adjacent to the transverse process.

Various types of screws, hooks, and clamps have been used for attaching corrective spinal instrumentation to selected portions of a patient's spine. Examples of pedicle screws and other types of attachments are illustrated in U.S. Pat. Nos. 4,763,644; 4,805,602; 4,887,596; 4,950,269; and 5,129,388. Each of these patents is incorporated by reference as if fully set forth herein.

Often, spinal fixation may include rigid (i.e., in a fusion procedure) support for the affected regions of the spine. Such systems limit movement in the affected regions in virtually all directions (for example, in a fused region). More recently, so called “dynamic” systems have been introduced wherein the implants allow at least some movement of the affected regions in at least some directions, i.e. flexion, extension, lateral, or torsional. While at least some known dynamic spinal implant systems may work well for their intended purpose, there is always room for improvement.

### SUMMARY OF THE INVENTION

In accordance with one feature of the invention, a dynamic spinal rod is provided for use in an implant system that supports a spine. The spinal rod includes an elongate body to extend along the length of the spine in use, the elongate body having a pair of anchor portions joined by an intermediate portion defining a longitudinal axis.

According to one feature, each of the anchor portions is configured for attachment by an anchoring system to a vertebra and/or to receive a connection for another component of a spinal implant system.

As one feature, the intermediate portion is configured to provide a first bending stiffness that allows an initial range of spinal flexion/extension and a second bending stiffness that restricts spinal flexion/extension beyond the initial range.

In one feature, the intermediate portion is configured to have a lower bending moment of inertia through a predetermined initial range of spinal bending and a higher bending moment of inertia beyond the initial range of spinal bending.

According to one feature, the intermediate portion has an outer surface and a groove in the outer surface having a pair of side walls, the side walls spaced from each other throughout the initial range and contacting each other beyond the initial range.

As one feature, the groove is a helical groove centered on the longitudinal axis.

In accordance with one feature, the outer surface tapers inward towards the longitudinal axis.

In one feature, the groove is one of plurality of transverse grooves.

According to one feature, the intermediate portion has a transverse cross section that varies in the longitudinal direction.

As one feature, each of the portions has a cylindrical shape.

In accordance with one feature of the invention, a system is provided for supporting a spine. The system includes first and second dynamic spinal rods to be fixed on laterally opposite sides of a spine.

Other features, advantages, and objects for the invention will become apparent after a detailed review of the entire specification, including the appended claims and drawings.

### BRIEF DESCRIPTION OF THE DRAWINGS

FIG. 1 is a somewhat diagrammatic representation of a spinal implant system in use and including a pair of dynamic spinal rods embodying the present invention;

FIGS. 2-5 depict various embodiments of dynamic rods for use in the system of FIG. 1, with FIG. 5 being a section view taken along line 5-5 in FIG. 4; and

FIG. 6 is similar to FIG. 1, but shows yet another embodiment of the dynamic spinal rods of the present invention.

### DETAILED DESCRIPTION OF THE PREFERRED EMBODIMENT

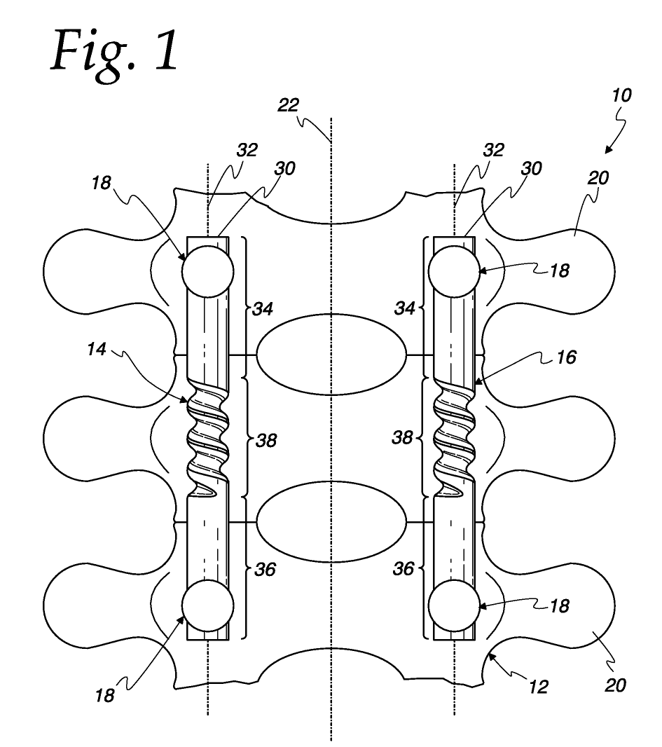

With reference to FIG. 1, a fixation or implant system 10 for supporting a spinal column 12 includes a pair of dynamic spinal rods 14 and 16 that are fixed on laterally opposite sides of the spine 12 by anchor systems 18 that connect the rods 14,16 to selected vertebra 20 of the spine 12. The components of the system 10 are preferably made from a suitable biocompatible material, such as titanium or stainless steel or other suitable metallic material, or ceramic, polymeric, or composite materials.

The system 10 is designed to allow a limited initial range of spinal bending, preferably flexion/extension motion, with the limited initial range of spinal bending preferably being sufficient to assist the adequate supply of nutrients to the disc in the supported portion of the spine 12. In this regard, while the range of bending may vary from patient to patient. Movement beyond the initial range of motion is restricted by the system 10 so as not to defeat the main purpose of the fixation system 10.

The system 10 is installed posterior to the spine 12, typically with the rods 14 and 16 extending parallel to the longitudinal axis 22 of the spine 12 lying in the mid-sagittal plane. It should be understood that while only two of the rods 14,16 are shown, the system 10 can include additional rods positioned further superior or inferior along the spine, with the additional rods being dynamic rods such as the rods 14 and 16, or being conventional non-dynamic or rigid rods. It should also be understood that the system 10 may also include suitable transverse rods or cross-link devices that help protect the supported portion of the spine 12 against torsional forces or movement. Some possible examples of suitable cross-link devices are shown in co-pending U.S. patent application Ser. No. 11/234,706, filed on Nov. 23, 2005 and naming Robert J. Jones and Charles R. Forton as inventors (the contents of this application are incorporated fully herein by reference). Other known cross-link devices or transverse rods may also be employed. Preferably, the rods 14 and 16 have sufficient column strengthen rigidity to protect the supported portion of the spine against lateral forces or movement.

Each of the rods 14,16 preferably has an elongate body 30 extending along a longitudinal axis 32 in an un-deformed state, with the body 30 having an integral or unitary construction formed from a single piece of material. While a single piece construction is preferred, in some applications it may be desirable for the body 30 to be made from a multiple piece construction.

The body 30 has a pair of anchor or connection portions 34 and 36 joined by an intermediate portion 38. Each of the anchor 34 and 36 is configured for attachment by a suitable anchoring system 18 to a vertebra 20, such as shown in FIG. 1, and/or to receive a connection for another component of a spinal implant system, such as, for example, a cross-link connection 19 such as shown in FIG. 6. In this regard, as is typical of spinal rods, the anchor portions 34 and 36 preferably are solid with a uniform cylindrical shape that is compatible with a variety of anchoring systems 18 and/or connections. However, other configurations are possible, such as, for example, solid prismatic shaped rod portions or elliptical shape or helical shape.

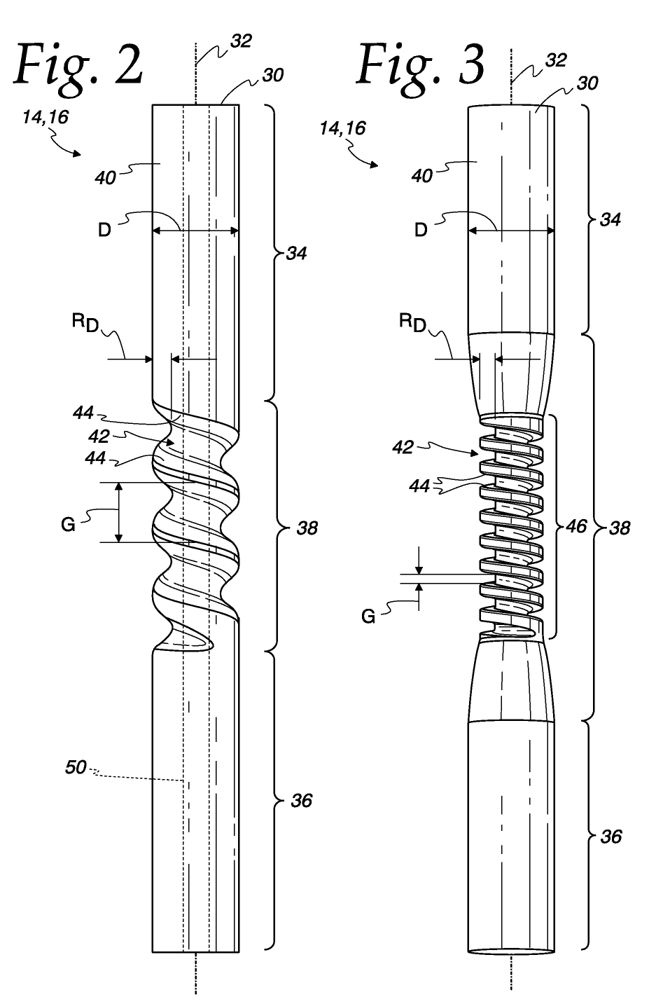

The intermediate portion 38 provides the “dynamic” or flexing capability for the rod 14,16 and is configured to provide a bending stiffness or a spring rate that is non-linear with respect to the bending displacement of the rod 14,16. This is intended to more closely mimic the ligaments in a normal stable spine which are non-linear in nature. The non-linear bending stiffness of the rods 14 and 16 is intended to allow the limited initial range of spinal motion and to restrict or prevent spinal motion outside of the limited initial range. In preferred embodiments, the non-linear bending stiffness is produced by configuring the intermediate portion 38 to provide a first bending stiffness that allows the initial range of spinal bending and a second bending stiffness that restricts spinal bending beyond the initial range of spinal motion. A preferred construction to achieve the first and second bending stiffnesses is to configure the intermediate portion 38 to have a lower bending moment of inertia I (sometimes referred to as the second moment of inertia or the area moment of inertia) through the initial range of spinal motion and a higher bending moment of inertia beyond the initial range of spinal motion. FIG. 2-4 show three possible embodiments for the rods 14,16 having intermediate sections 38 that achieve the foregoing.

The rod 14,16 shown in FIG. 2 has a cylindrical outer surface 40 that is interrupted by a helical groove 42 that is centered on the longitudinal axis 32 and extends over the length of the intermediate section 38. The outer surface 40 has a diameter D. The groove 42 has radial depth RD, and a pair of side walls 44 that are spaced by a longitudinal distance G in the un-deformed state of the intermediate section 38. Because the groove depth RD reduces the diameter of the intermediate section 38 at each transverse cross section along the helical groove 42, the bending moment of inertia I of the intermediate section 38 is reduced in comparison to the bending moment of inertia of the remainder of the rod 14,16, which results in a lower or reduced bending stiffness for the intermediate section 38 in comparison to the anchor sections 34 and 36. However, bending of the rod 14,16 will decrease the gap G between the walls 44 on the compression side of the rod 14,16 until the sidewalls 44 come into contact with each other after the initial range of bending has taken place. When the sidewalls 44 are in contact, the bending stiffness of the intermediate section 38 closely approaches or is essentially equal to the bending stiffness of each of the anchor sections 34 and 36 because the bending moment of inertia is increased due to the contacting walls 44. In this regard, it is preferred that the lower bending stiffness be selected so as to allow flexion/extension of the spine 12 without undue effort or discomfort to the patient, and that the higher bending stiffness be essentially rigid in the context of the patient's ability to bend the spine 12 beyond the initial range.

The range of initial bending will be dependent upon the ratio of the gap G to the diameter D, with smaller ratios producing a smaller range of initial bending and larger ratios producing a larger range of initial bending. Furthermore, the range of initial bending will be dependent upon the number of gaps provided over the length of the intermediate section, with the range of initial bending increasing with an increased number of gaps. By careful selection of the ratio of G/D and the number of gaps provided over the length of the intermediate section 38, the desired initial range of bending for the rod 14,16 and for the spine 12 can be achieved.

In addition to the above discussed changes in the geometry of the groove 42 in order to achieve the desired initial range of bending, it will be appreciated by those skilled in the art that changes in the geometry of the groove 42, and side walls 44, can also be made in order to manipulate the bending stiffness and bending moment of inertia, both in the initial range of bending and beyond the initial range of bending. For example, changes in the angle of the side walls 44, the depth RD of the groove 42, and blend radii, will all have an effect.

It should be appreciated that the helical groove 42 provides an asymmetric bending stiffness about the longitudinal axis 32, thereby allowing the rod 14,16 to be implanted without concern for a particular angular orientation of the rod 14,16 about its longitudinal axis 32 with respect to the spine 12.

The rod 14,16 of FIG. 3 is similar to the rod 14,16 of FIG. 2 but differs in that intermediate portion 38 has been tapered inward so that a central length 46 of the intermediate section 38 has a reduced diameter, and in that the helical groove 42 has a finer pitch which produces a smaller value for the gap G and a larger number of reduced transverse cross sections in comparison to the rod 14,16 of FIG. 2. Thus, it will be appreciated by those skilled in the art that the rod 14,16 of FIG. 3 has a lower bending stiffness and a lower bending moment of inertia through the initial range of bending than the rod 14,16 of FIG. 2.

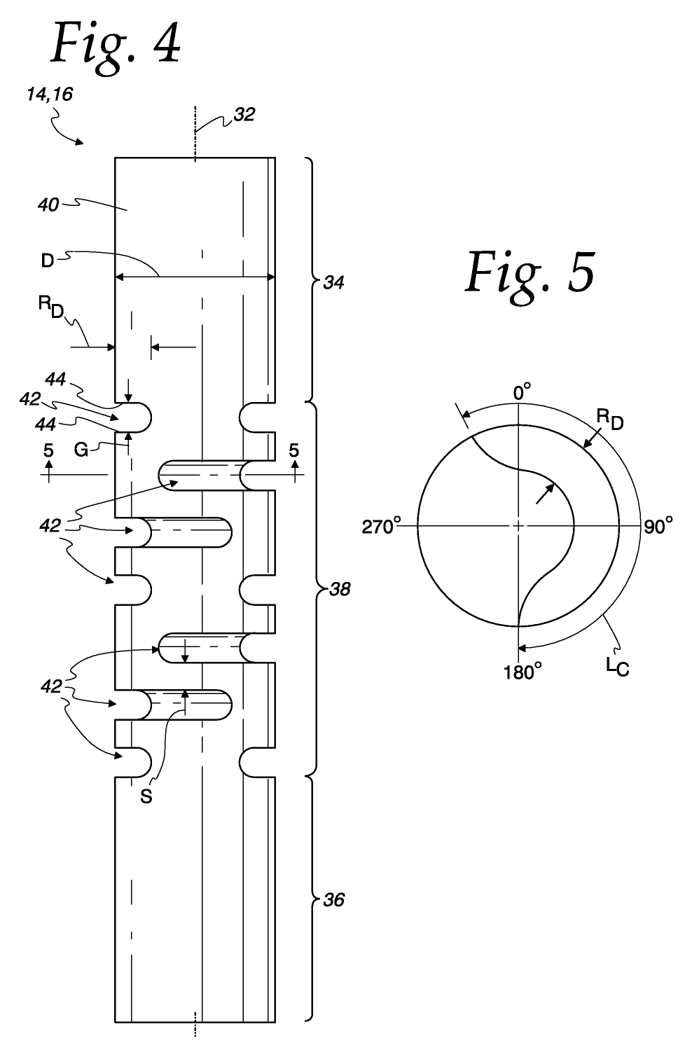

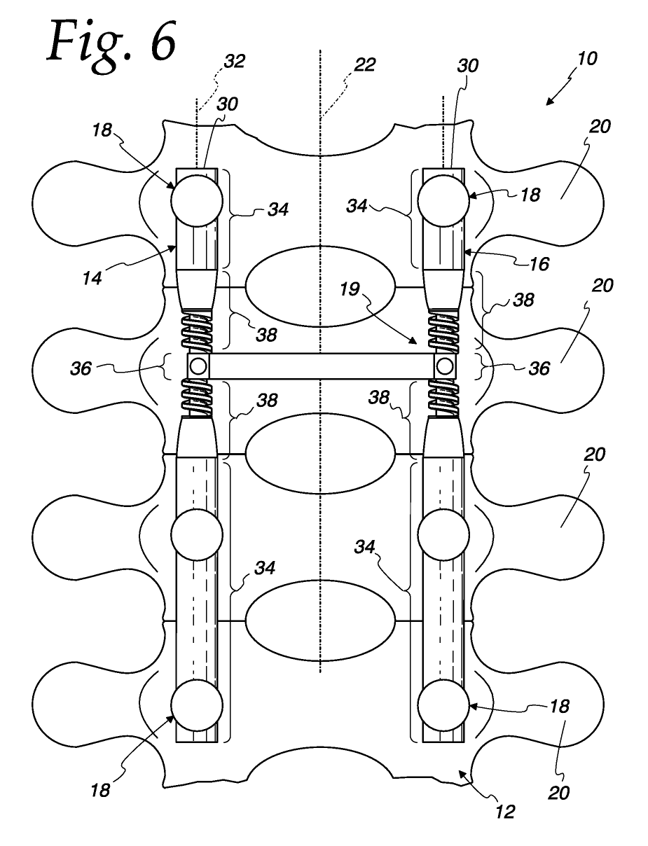

FIGS. 4 and 5 show yet another alternative for the rod 14,16 wherein a plurality of transverse, annular grooves 42 are provided rather than the single helical groove 42 of FIGS. 2 and 3. The grooves 42 are spaced longitudinally over the length of the intermediate section 38, with the longitudinal spacing S from one groove 42 to the next 42 either being consistent throughout the intermediate section 38 as shown in FIG. 4, or varying throughout the section 38. Furthermore, in the illustrated embodiment, the grooves 42 extend only partially around the circumference of the intermediate section 38. In this regard, it is preferred that the angular position of the grooves 42 be “clocked” or rotated about the axis 32 from groove to groove to provide an asymmetric bending stiffness about the longitudinal axis 32, thereby allowing the rod 14,16 to be implanted without concern for a particular angular orientation of the rod 14,16 about its longitudinal axis 32 with respect to the spine 12. For example, the groove 42 shown in FIG. 5 extends from about 335° to 180°. The next groove down from the groove 42 of FIG. 5 will extend from 0° to about 205°. Additionally, the circumferential length L_{C} of each groove 42 can be varied. For example, the groove 42 immediately above the groove 42 of FIG. 5 may extend 270° from the 225° position to the 135° position. It will be appreciated that there are a number of possibilities for the groove to groove clocking, and the clocking will depend on a number of factors, including, for example, the number of transverse grooves 42 and how far around the circumference of the intermediate section 38 each groove 42 extends.

While the transverse grooves 42 shown in FIG. 3 extend only partially around the circumference of the intermediate section 38, in some applications it may be desirable for the grooves 42 to extend completely around the circumference. Additionally, while the grooves 42 have been shown as annular, in some embodiments it may be desirable for the grooves to have a non-annular configuration, such as, for example, planar grooves 42.

FIG. 6 shows a number of possible variations for the dynamic rods 14,16. As one variation, one of the anchor sections 36 of each rod is connected by a cross-link device 19. Another variation is the inclusion of a second intermediate section 38 on the opposite end of the anchor section 36 of each rod together with a second anchor section 34. Yet another variation is that the second anchor section 34 has sufficient length to be anchored to two of the vertebra 20 with anchors 18. It should be appreciated that these illustrated variations are but a few of the many possible for the dynamic rods 14,16 shown in FIGS. 1-6. For example, the second anchor section 34 could be lengthened to allow anchoring to any number of vertebra 20. As yet another example, while the dynamic rods 14,16 of FIGS. 3 and 6 are shown with intermediate sections 38 that taper inwardly from the anchor sections 34,36, in some embodiments it may be desirable for the intermediate sections 38 to taper outwardly to a larger diameter than the corresponding anchor sections 34,36. Furthermore, as another example, it may desirable for each of the sections 34,38,36 of the dynamic rods 14,16 to have different outer diameters than the other sections. As another example, it should be appreciated that while FIG. 6 shows the intermediate sections 38 as being tapered, any of the intermediate sections 38 described and shown herein, including those described in connection with FIGS. 1, 2, 4 and 5 can be utilized as one or more of the intermediate sections 38 shown in FIG. 6. As yet another alternative, in any of the previously described embodiments, an elongate hole may extend through the entire length of the dynamic rod 14,16 centered on the axis 32, such as shown by the longitudinal hole 50 in FIG. 2, as yet another means of achieving the desired initial bending stiffness/bending moment of inertia. In this regard, the diameter of the longitudinal hole 50 could be enlarged in the area of the intermediate section 38 in order to provide a lower bending stiffness/bending moment of inertia. It should also be appreciated that, as with conventional non-dynamic rods, the dynamic rods 14,16 can be permanently deformed or bent to match a desired curvature of the corresponding portion of the spine 12 and that this permanent deformation can either be preformed by the manufacturer or custom formed by the surgeon during a surgical procedure.

The system 10 according to the invention may be used in minimally invasive surgery (MIS) procedures or in non-MIS procedures, as desired, and as persons of ordinary skill in the art who have the benefit of the description of the invention understand. MIS procedures seek to reduce cutting, bleeding, and tissue damage or disturbance associated with implanting a spinal implant in a patient's body. Exemplary procedures may use a percutaneous technique for implanting longitudinal rods and coupling elements. Examples of MIS procedures and related apparatus are provided in U.S. patent application Ser. No. 10/698,049, filed Oct. 30, 2003, U.S. patent application Ser. No. 10/698,010, filed Oct. 30, 2003, and U.S. patent application Ser. No. 10/697,793, filed Oct. 30, 2003, incorporated herein by reference. It is believed that the ability to implant the system 10 using MIS procedures provides a distinct advantage.

Persons skilled in the art may make various changes in the shape, size, number, and/or arrangement of parts without departing from the scope of the invention as described herein. In this regard, it should also be appreciated that the various relative dimensions of each of the portions 34, 36, and 38, and of the grooves 42 are shown in the figures for purposes of illustration only and may be changed as required to render the system 10 suitable for its intended purpose.

Various other modifications and alternative embodiments of the invention in addition to those described herein will be apparent to persons of ordinary skill in the art who have the benefit of the description of the invention. Accordingly, the description, including the appended drawings, is to be construed as illustrative only, with the understanding that preferred embodiments are shown.

1. A dynamic spinal rod for use in an implant system that supports a spine, the spinal rod comprising:
an elongate body to extend along the length of the spine in use, the elongate body having a pair of anchor portions joined by an intermediate portion defining a longitudinal axis; and
the intermediate portion configured to provide a first bending stiffness that allows an initial range of spinal bending and a second bending stiffness that restricts spinal bending beyond the initial range.

2. The rod of claim 1 wherein the intermediate portion has an outer surface and a groove in the outer surface having a pair of side walls, the side walls spaced from each other throughout the initial range and contacting each other beyond the initial range.

3. The rod of claim 2 wherein the groove is a helical groove centered on the longitudinal axis.

4. The rod of claim 3 wherein the outer surface tapers inward towards the longitudinal axis.

5. The rod of claim 2 wherein the groove is one of plurality of transverse grooves.

6. The rod of claim 1 wherein the intermediate portion has a transverse cross section that varies in the longitudinal direction.

7. The rod of claim 1 wherein each of the portions has a cylindrical shape.

8. The rod of claim 1 wherein each of the anchor portions is configured for attachment by an anchoring system to a vertebra and/or to receive a connection for another component of a spinal implant system.

9. A dynamic spinal rod for use in an implant system that supports a spine, the spinal rod comprising:
an elongate body to extend along the length of the spine in use, the elongate body having a pair of anchor portions joined by an intermediate portion defining a longitudinal axis; and
the intermediate portion configured to have a lower bending moment of inertia through a predetermined initial range of spinal bending and a higher bending moment of inertia beyond the initial range of spinal bending.

10. The rod of claim 9 wherein the intermediate portion has an outer surface and a groove in the outer surface having a pair of side walls, the side walls spaced from each other throughout the initial range and contacting each other beyond the initial range.

11. The rod of claim 10 wherein the groove is a helical groove centered on the longitudinal axis.

12. The rod of claim 11 wherein the outer surface tapers inward towards the longitudinal axis.

13. The rod of claim 10 wherein the groove is one of plurality of transverse grooves.

14. The rod of claim 9 wherein the intermediate portion has a transverse cross section that varies in the longitudinal direction.

15. The rod of claim 9 wherein each of the portions has a cylindrical shape.

16. The rod of claim 9 wherein each of the anchor portions is configured for attachment by an anchoring system to a vertebra and/or to receive a connection for another component of a spinal implant system.

17. A system for supporting a spine, the system comprising first and second dynamic spinal rods to be fixed on laterally opposite sides of a spine, each of the rods comprising:
an elongate body to extend along the length of the spine in use, the elongate body having a pair of anchor portions joined by an intermediate portion defining a longitudinal axis; and
the intermediate portion configured to provide a first bending stiffness that allows an initial range of spinal flexion/extension and a second bending stiffness that restricts spinal flexion/extension beyond the initial range.

18. The system of claim 17 wherein the intermediate portion has an outer surface and a groove in the outer surface having a pair of side walls, the side walls spaced from each other throughout the initial range and contacting each other beyond the initial range.

19. The system of claim 18 wherein the groove is a helical groove centered on the longitudinal axis.

20. The system of claim 19 wherein the outer surface tapers inward towards the longitudinal axis.

21. The system of claim 19 wherein the groove is one of plurality of transverse grooves.

22. The rod of claim 17 wherein each of the anchor portions is configured for attachment by an anchoring system to a vertebra and/or to receive a connection for another component of a spinal implant system.

23. A system for supporting a spine, the system comprising first and second dynamic spinal rods to be fixed on laterally opposite sides of a spine, each of the rods comprising:
an elongate body to extend along the length of the spine in use, the elongate body having a pair of anchor portions joined by an intermediate portion defining a longitudinal axis; and
the intermediate portion configured to have a lower bending moment of inertia through a predetermined initial range of spinal bending and a higher bending moment of inertia beyond the initial range of spinal bending.

24. The system of claim 23 wherein the intermediate portion has an outer surface and a groove in the outer surface having a pair of side walls, the side walls spaced from each other throughout the initial range and contacting each other beyond the initial range.

25. The system of claim 24 wherein the groove is a helical groove centered on the longitudinal axis.

26. The system of claim 25 wherein the outer surface tapers inward towards the longitudinal axis.

27. The system of claim 24 wherein the groove is one of plurality of transverse grooves.

28. The rod of claim 23 wherein each of the anchor portions is configured for attachment by an anchoring system to a vertebra and/or to receive a connection for another component of a spinal implant system.
